(19) United States
(12) Plant Patent Application Publication
(10) Pub. No.: US 2015/0282400 P1
(43) Pub. Date: Oct. 1, 2015

(54) Apple tree named 'Galafab'

(50) ;

(71) Applicant: Lionel Fabre, Entraigues (FR)

(72) Inventor: Lionel Fabre, Entraigues (FR)

(21) Appl. No.: 13/999,856

(22) Filed: Mar. 28, 2014

(51) Int. Cl. A01H 5/00

(52) U.S. Cl. PLT/162

(57) ABSTRACT

‘Galafab’ is a new and distinct variety of apple tree (Malus domestica Borkh). A mutation of ‘Gala’ (not patented), ‘Galafab’ is characterized by its intense red overcolor.

### CROSS REFERENCE TO RELATED APPLICATIONS

None

### LATIN NAME

Malus domestica Borkh

### COMMON NAME

Apple

### VARIETY DENOMINATION

‘Galafab’

### BACKGROUND OF THE VARIETY

‘Galafab’ is a new and distinct variety of apple tree Malus domestica Borkh. This new variety is a mutation of ‘Gala’ (not patented), and is characterized by its intense red overcolor.

‘Galafab’ was first observed at Athens les Paluds, France in 2008, and was asexually reproduced by grafting at the same location in 2009. The variety has been observed to remain true to type over successive asexually propagated generations.

### BRIEF DESCRIPTION OF THE VARIETY

‘Galafab’ was first selected for its uniquely colored fruit. It is distinguished from other known Gala-type varieties by its intense red overcolor, as set forth in the table below:

| Characteristic | ′Galafab′ | ′Gala′ |
|---|---|---|
| Relative area of over color | Very large | Medium |
| Pattern of over color | Solid flush with strongly | Large stripes |
| | defined stripes | |

### BRIEF DESCRIPTION OF THE PHOTOGRAPHS

FIGS. 1 and 2 show trees of ‘Galafab’; and,

FIGS. 3 and 4 show fruit and leaves of ‘Galafab’.

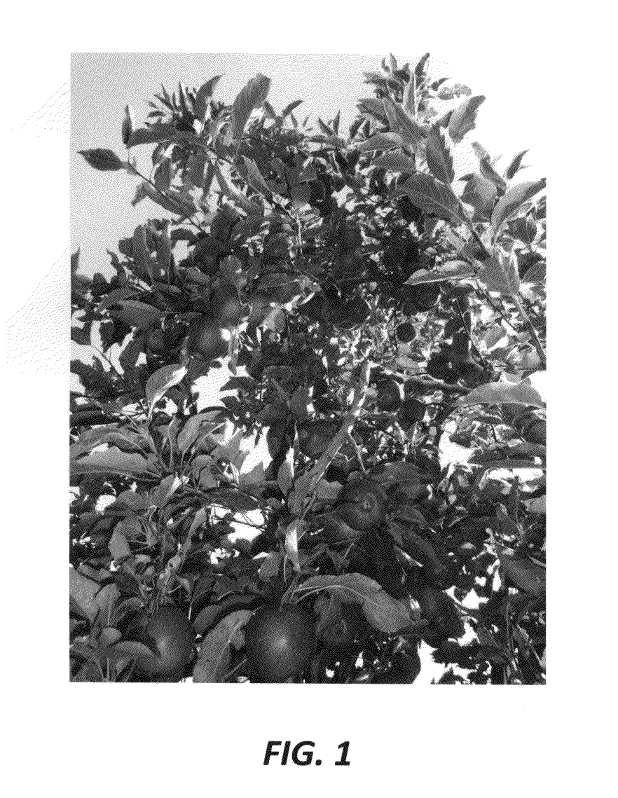

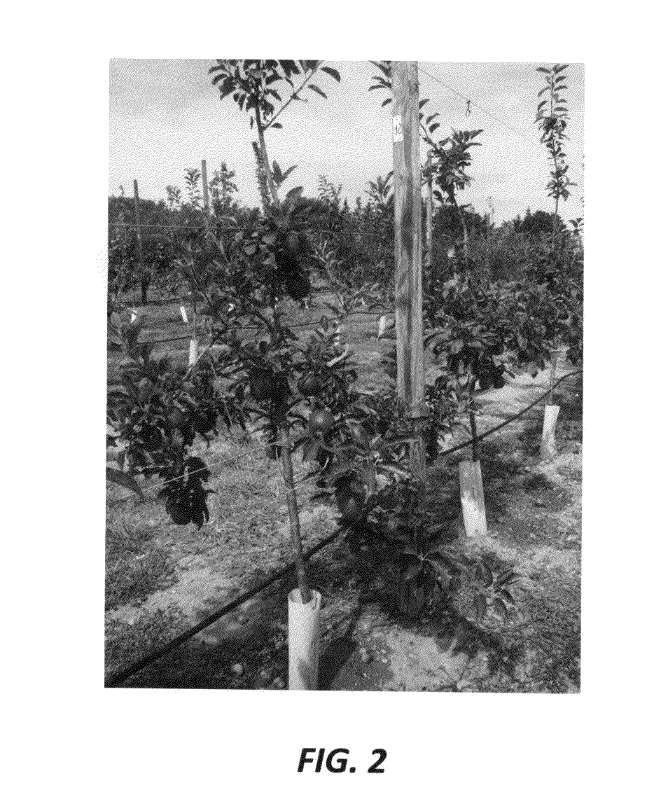

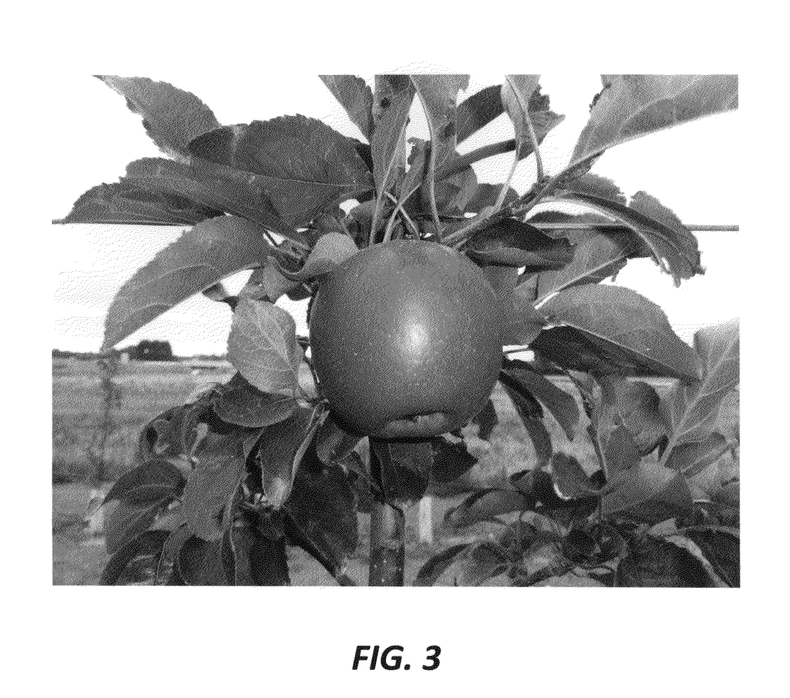

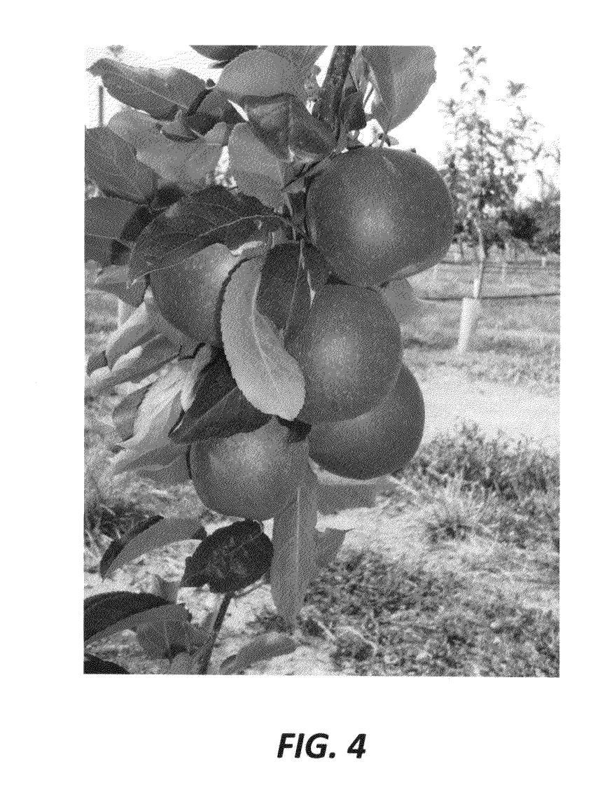

### DETAILED BOTANICAL DESCRIPTION OF THE VARIETY

The following-detailed botanical description is based on observations made during the 2013 growing season at Béziers, France, of trees planted in 2010 and grown on ‘M9 EMLA’ rootstock (not patented). It should be understood that the characteristics described will vary somewhat depending upon cultural practices and climatic conditions, and will vary with location and season. Quantified measurements are expressed as an average of measurements taken from a number of individual plants of the new variety. The measurements of any individual plant or any group of plants of the new variety may vary from the stated average.
Tree:
   Vigor.—Medium.
   Type.—Ramified.
   Habit.—Spreading.
   Height.—Medium small.
   Trunk diameter (at 30 cm above the graft).—Medium (16 to 20 cm).
   Bark texture.—Smooth.
   Bark coloration.—Light grey.
Branch (fruiting branches located at around 1 m above the graft union):
   Length.—Medium.
   Diameter.—Medium.
   Crotch angle.—Wide.
   Bark color.—Light Grey.
   Lenticel length.—Medium long.
   Lenticel color.—Black.
   Number of lenticels per cm².—Numerous.
One year old shoot:
   Length.—30 to 40 cm.
   Color.—Grey.
   Pubescence.—Weak.
   Thickness.—Medium.
   Internode length.—Medium.
   Number of lenticels per cm².—Many.
Flower buds:
   Shape.—Triangular, pointed.
   Length.—1.8 to 2.4 cm.
   Diameter.—1.6 to 2.0 cm.
   Color.—Green, with white pubescence.
Flowers:
   Diameter of fully open flower.—Medium (4.0 to 4.5 cm).
   Relative position of petal margin.—Free.
   Number per cluster.—6.
   Date of first bloom.—April 1^{st}.
   Date of full bloom.—April 5^{th}.
   Pollination requirement.—similar to Gala.
Petals:
   Number per flower.—5 (simple row).
   Shape.—Ovoid.
   Length.—1.8 cm.
   Width.—1.4 cm.
   Apex.—Round.
   Base.—Elongated.
   Margin.—Smooth.
   Coloration of upper surface.—White.
   Coloration of lower surface.—White with pink.
Pistils:
   Size.—1.1 cm.
   Color.—Green/yellow.
Stigma:
   Size.—Prominent.
   Color.—Yellow.
Style:
   Size.—1.1 cm.
   Color.—Green/yellow (pubescent).
Ovary:
   Size.—0.3×0.3 cm.
   Color.—Green.
Anthers:
   Quantity.—18 (one distinct whorl).
   Size.—0.6 cm to 0.8 cm.
   Presence of pollen.—Yes.
   Color of pollen.—Yellow.
Pedicel of flower:
   Length.—2.2 to 2.6 cm.
   Diameter.—0.1 to 0.2 cm.
   Color.—Green.
Sepals:
   Quantity.—5.
   Coloration.—Green (pubescent).
   Shape.—Elongated/pointed.
   Margin.—Pointed.
Leaves:
   Shape.—Elliptic.
   Length.—Long.
   Width.—Medium.
   Length/width ratio.—Large.
   Blade margin.—Crenate, finely serrate.
   Apex.—Pointed.
   Base shape.—Crenate.
   Color of upper surface.—Olive green.
   Color of lower surface.—Green.
   Attitude in relation to shoot.—Upwards.
Petiole:
   Length.—Medium.
   Diameter.—Unavailable Data.
   Coloration.—Unavailable Data.
Fruit:
   Quantity per cluster.—.
   Diameter.—Medium (6.5 to 7.5 cm).
   Weight.—Medium (167 g to 210 g).
   Ratio of height to width.—1:1.
   General shape in profile.—Truncate conical.
   Position of maximum diameter.—Toward stalk.
   Ribbing.—Medium.
   Crowning at calyx end.—Medium.
   Size of eye.—Medium.
   Aperture of eye.—Closed.
   Length of sepal.—Long.
   Bloom of skin.—Absent or very weak.
   Greasiness of skin.—Absent or very weak.
   Background color of skin.—Yellow.
   Over color of skin.—Red.
   Amount of over color.—90% to 100%.
   Intensity of over color.—Intense.
   Pattern of over color.—Solid flush with strongly defined stripes.
   Progression of over color before harvest.—30 days — 50% to 75% drab red; 15 days — 75% to 100% drab red; 5 days — 90% to 100% bright red.
   Amount of russet around stalk cavity.—Low.
   Amount of russet on cheeks.—Absent or very low.
   Area of russet around eye basin.—Absent or very low.
   Length of stalk.—Long.
   Thickness of stalk.—Medium.
   Depth of stalk cavity.—Deep.
   Width of stalk cavity.—Medium.
   Depth of eye basin.—Deep.
   Width of eye basin.—Medium.
   Firmness of flesh.—Medium.
   Flesh texture.—Juicy, fine, crisp.
   Aroma.—Aromatic, Gala-type aroma.
   Juiciness.—Important.
   Brix.—.
   Flesh coloration.—Yellowish, satin white.
   Stem coloration.—Yellow brown.
Seeds:
   Quantity per fruit.—7 to 9.
   Shape.—Unavailable Data.
   Coloration .—Dark brown.
Harvest:
   Time for harvest.—10 August (2013 growing season).
   Number of picks.—3.
   Amount of fruit produced per tree per harvest.—68 to 118.
   Weight.—176 grams.
Disease resistance/susceptibility: Same as Gala
Market use: Fresh Market

1. I claim a new and distinct apple tree named ‘Galafab’ substantially as illustrated and described herein.
